(19) United States
(12) Patent Application Publication
(10) Pub. No.: US 2011/0262506 A1
(43) Pub. Date: Oct. 27, 2011

(54) TOPICAL COMPOSITIONS CONTAINING COATED ACTIVE AGENTS

(75) Inventors: Ofer Toledano, Kfar-Saba (IL); Daniela Mavor, Tel Aviv (IL); Eli Drori, Tel Aviv (IL); Maya Erlich, Ness Ziona (IL); Sharon Hershkovitch, Modiin (IL)

(73) Assignee: SOL-GEL TECHNOLOGIES LTD., Ness Ziona (IL)

(21) Appl. No.: 13/126,288

(22) PCT Filed: Jan. 5, 2010
(86) PCT No.: PCT/IL2010/000010

§ 371 Date: Jun. 27, 2011

Related U.S. Application Data
(60) Provisional application No. 61/193,892, filed on Jan. 5, 2009.

(51) Int. Cl. A61K 8/02; A61P 17/10; A61P 31/00; A61P 29/00; A61P 17/04; A61B 19/02; A61P 17/08; A61P 17/00; A61P 35/00; A61P 17/16; A61Q 19/02; A61Q 19/00; A61K 9/00; A61P 17/06

(52) U.S. Cl. 424/401; 424/400; 206/438

(57) ABSTRACT

Provided is a multi component topical system including at least one component including at least one first active agent coated by metal oxide; and at least one skin treating component free of the active agent. Further provided are methods of using the multi component topical system and kits including them.

### FIELD OF THE INVENTION

This invention relates to systems and kits for topical and dermatological uses.

### BACKGROUND OF THE INVENTION

For the past 40 years, Benzoyl Peroxide (BPO) has been successfully used in treatment of skin acne. It is considered the most effective topical treatment intended for eradication of Propionibacterium acnes (p. acnes). In contrast to antibiotics, BPO does not cause bacterial resistance. In addition, it is a keratolytic compound that removes the top layer of skin so that skin cells are less likely to clog pores and enhances desquamation of the follicular plug. Some of the side effects of BPO use are dryness and irritation to skin portions in contact with it.

Many individual skin treatment products comprising BPO are known and sold in the market. Additionally, kits comprising BPO together with other skin conditioning products are also known. Patients suffering from acne, often suffer from adverse effects such as dryness, irritation, erythema, scaly skin as a result of the BPO treatment. Conventional non-encapsulated BPO crystals in known BPO-based products dissolve rapidly in the skin sebum generating high degree of adverse effects. In addition such non-encapsulated BPO crystals provide short anti-bacterial activity, whereas encapsulated BPO does not dissolve rapidly and maintains prolonged therapeutic concentration of BPO at the target desired site. In addition to frequent consumer migration to competitive products, these adverse symptoms and decreased efficacy cause patients to often change and even abandon such BPO products and switch to antibiotics.

It is highly advantageous to provide a coated skin treatment system, especially for the treatment of acne, wherein such adverse effects are prevented and wherein the combination of system components (with and without an active agent such as for example BPO) together with administration regimen provide superior treatment results as compared to conventional known BPO kits.

### SUMMARY OF THE INVENTION

The present invention is based on the finding that a multi component topical system comprising components, which comprise an active agent coated by metal oxide and a skin treating component free of the active agent provides a surprisingly beneficial, unexpectedly enhanced and more effective treatment of skin conditions as compared with known systems wherein the active agent is uncoated. The enhanced and improved effect of the multi component topical system of the invention was found to be greater than the effect of each of the system's components taken separately.

Upon contact with the skin, the active agent is extracted efficiently from within the coating into the sebum, in a delayed and prolonged manner, and finds its way preferentially to the target treatment area (for example to the sebaceous glands where Propionibacterium acnes resides). Application of the metal oxide coated active agent does not expose the skin to a burst of the active agent concentration which provides markedly less local adverse effects and permits use of a much higher concentrated active agent and for a more efficient and prolonged effect. The use of such metal oxide coated active agent with the skin treating component free of said active agent provides for a much more efficient, long-term and potent treatment of skin or mucosal membrane conditions and disorders as compared with free active agent containing kits or systems.

According to the first aspect of the invention there is provided a multi component topical system comprising: at least one component comprising at least one first active agent coated by metal oxide; and at least one skin treating component being free of said active agent.

The term “multi component topical system” as used herein is meant to encompass an arrangement or collection of at least two components having the same or different contents, for topical and/or dermatological use. In some embodiments, the system comprises 2, 3, 4, 5, 6, 7,8, 9 or 10 components having the same or different contents encompassed in the same or different vessels or containers, having the same or different carriers and active agents for topical and/or dermatological use.

It should be noted that a system of the invention may further comprise a component which is not directly used by a subject, but rather is used for the preparation of another component of the system, e.g., for dilution purposes, optional addition of a sensitive agent to a component and so forth.

It should be noted that the system of the invention is intended for topical and/or dermatological use on any type of skin area, being an exterior exposed area (such as for example areas of the skin, scalp, hair, and nails), an interior skin area such as a mucosal membrane (such as for example mucosal membrane around and on the nostrils, the lips, the ears, the genital area, and the anus) or any vicinal areas in close proximity with the treated skin or mucosal membrane areas wherein said system components may reach via any kind of diffusion mechanisms to a skin area or mucosal membrane.

In some embodiments, a system of the invention comprises at least one further component, each of said at least one further components comprising at least one further active agent coated by metal oxide.

In other embodiments, said at least one first active agent and at least one second active agent are the same. In still other embodiments, said at least one first active agent and at least one second active agent are different.

In further embodiments, said at least one further component is a second component comprising at least one second active agent coated by metal oxide.

Yet still, in some additional embodiments, said at least one first active agent and at least one further active agent are the same. In another embodiment, the two are different.

It should be noted that the terms “same” or “different” with respect to the contents of a component of a system of the invention are meant to refer to at least one typical characterization of the denoted component such as, but not limited to, the nature of the active agent, its concentration, the additional at least one additive comprised therein and their concentration, and so forth.

The term “active agent” as used herein refers to any kind of pharmacologically or cosmetically active agent. In some embodiments of the present invention, said at least one active agent coated by metal oxide is selected from the following non-limiting list: an anti fungal agent, an anti bacterial agent, an anti viral agent, an anti inflammatory agent, an anti pruritic agent, an anti psoriatic agent, an anti seborrhea, an anti acne agent, an anti rosacea agent, an anti skin cancer agent, an anti photoaging agent, a skin whitening/bleaching agent, an anti skin redness agent and any combinations of the above. In some embodiments, said active agent is an anti acne agent. In further embodiments, said anti acne agent is benzoyl peroxide or a mixture thereof with at least one additional active agent as defined.

As used herein, the term “active agent coated by metal oxide” or any of its lingual equivalents is meant to encompass an active agent(s) as disclosed hereinabove and for the purposes disclosed herein being coated by a metal oxide coating, wherein an active agent or particulates of an active agent are encapsulated within a core of a metal oxide coating. It should be understood that the metal oxide coating may be obtained in any of the processes known to a person skilled in the art, be it a process having a single processing step or a multi-step, repeated or circular processes. A process for obtaining a metal oxide coating of an at least one active agent should enable the formation and growth of a coat or coats of a metal oxide on the active agent particulates, with the ability of fine control of the width of the obtained coat. This is particularly advantageous for topical and/or dermatological uses where the active ingredient should be isolated, from its surroundings with an ability to be gradually released through the metal oxide coating. It is particularly advantageous to use a metal oxide coating process which enables fine tuning and control of the thickness of the metal oxide coating.

Examples of metal oxide coating processes may be found in U.S. Pat. No. 6,303,149, U.S. Pat. No. 6,238,650, U.S. Pat. No. 6,468,509, U.S. Pat. No. 6,436,375, US2005037087, US2002064541, WO00/09652, WO00/72806, WO01/80823, WO03/03497, WO03/039510, WO00/71084, WO05/009604, WO04/81222, EP0934773, U.S. Pat. No. 6,337,089, EP 0 941 761, U.S. Pat. No. 6,251,313, U.S. Pat. No. 4,931,362, GB2416524, U.S. Pat. No. 6,855,335, WO03/066209, WO98/31333, U.S. Pat. No. 6,495,352, U.S. Pat. No. 5,292,801, all of which are herein incorporated by reference.

In some embodiments, said metal oxide coating is selected from Silica, Titania, Alumina, Zirconia, ZnO, and mixtures thereof.

In the present invention the term “coating” and other lingual variations thereof, refer to a layer of metal oxide formed around a particle or particulate matter of the active agent. The layer or coating may a uniform coating or an incomplete coating. Particulates produced by the process of metal oxide coating, as will be further disclosed hereinbelow, comprise a core composed of a solid, water insoluble active agent particulates; said core is coated by a metal oxide coating; wherein said metal oxide coating is substantially non-amorphous and/or in a non-crystalline form.

In some embodiments, the coating is a composite of metal oxide and at least one adhering agent. Such adhering additive may be for example a polymer which interrupts the formation of continues regions of the metal oxide, thereby leading to the non-amorphous and non crystalline metal oxide form.

In a further aspect of the present invention there is provided a multi component topical system comprising: at least one component comprising at least one first active agent coated by a metal oxide selected to provide gradual release of said at least one first active agent; and at least one skin treating component being free of said active agent.

In this context it should be noted that a metal oxide capable of providing gradual release of at least one active agent coated thereby may endow the system of the invention with at least one of the following benefits: a) a reduced topical and/or dermatological side effect (such as for example irritation, skin or mucosal membrane sensitivity, development of unwanted rashes or pigmentation changes of the treated skin or mucosal membrane or any area in its vicinity, etc.) at any area of treated skin or mucosal membrane, which could be caused by any component of a system, b) an increased dispersability of the coated active agent, c) prevention of direct contact of the active agent and the skin, d) prevention or reduction in the crystal growth processes of the active agent, e) increased stability of the active agent either in storage, during use and upon application to the skin or mucosal membrane, f) increased compatibility of the coated active agent with other ingredients in a formulation of a component of a system of the invention, and g) a sustained and gradual release of an active agent coated by a metal oxide onto the treated skin.

In order to achieve the purpose of the invention, at least one component of a system of the invention comprises a skin treating component which is free of the same active agent which is coated by metal oxide and contained in at least one other component of the system of the invention. Notwithstanding, the skin treating component may comprise any kind of skin toning, cleaning, conditioning, improving, treating or exfoliating agent, agents capable of removing any kind of soil, dirt or any excessive material(s) (which may be derived from any external source(s) or any local source(s) such as for example emissions extracted from skin lesions) on the surface of the treated skin or mucosal membrane area. Some non-limiting examples of skin treating components are skin bracers or fresheners (may comprise 0-10% alcohol, water, and a humectant such as for example glycerine), skin tonics (may contain up to 20% alcohol, water and a humectant) or astringents (may contain 20-60% alcohol, antiseptic ingredients, water, and a humectant ingredient) or any combination thereof.

The skin treating component may also comprise at least one activeagent, which is different from the active agent disclosed, and which may yet be selected from the following non-limiting list: an anti fungal agent, an anti bacterial agent, an anti viral agent, an anti inflammatory agent, an anti pruritic agent, an anti psoriatic agent, an anti seborrhea, an anti acne agent, an anti rosacea agent, an anti skin cancer agent, an anti photoaging agent, a skin whitening/bleaching agent, an anti skin redness agent and any combinations of the above. The at least one active ingredient is not coated with a metal oxide coating. In some embodiments of the present application said at least one active agent comprised in said at least one skin treating component is optionally coated by a metal oxide.

It should be noted that each component of a system of the invention may be formulated for topical application in any formulation as known to a person skilled in the art. Such formulation may be selected from an ointment, a cream, a lotion, oil, an emulsion, a gel, a paste, a milk, an aerosol, a powder, a foam, a wash (such as for example a wash-on, a wash off formulation) and any combination thereof.

As used herein, a “wash-on” formulation encompasses a formulation comprising any cleansing vehicles applied to a surface or any combination thereof, which is generally applied to a surface of a skin or mucosal membrane in order to perform a cleaning function, and in addition at least a portion of the wash-on formulation remains on the surface of a skin or mucosal membrane to provide a further enhanced function. As used herein, a “wash-off” formulation encompasses a formulation comprising any cleansing vehicles applied to a surface or any combination thereof, which is generally applied to a surface of a skin or mucosal membrane which is at a later stage removed from the applied surface.

Exemplary forms of cleansing vehicles include, but are not limited to, liquid, bar, gel, foam, aerosol or pump spray, cream, lotion, stick, powder, or incorporated into a patch or a towelette. In addition, soapless cleansers may be used as well.

It should be noted that any component of a system of the invention may be incorporated in any kind of product form such as, but not limited to: a soap including liquid and bar soap, a shampoo, a hair conditioner, a shower gel, including an exfoliating shower gel, a foaming bath product (e.g. gel, soap or lotion), a milk bath, a soapless cleanser, including a gel cleanser, a liquid cleanser and a cleansing bar, moist towelletes, a body lotion, a body spray, mist or gel, bath effervescent tablets (e.g., bubble bath), a hand and nail cream, a bath/shower gel, a shower cream, a depilatory cream, a shaving product e.g. a shaving cream, gel, foam or soap, an after-shave, after-shave moisturizer, a sunscreen lotion, gel or oil, any kind of make-up product in a cream, lotion or cake form, and any combinations thereof, and any other products used for cleansing, post-cleansing, make-up application to the body, including the skin and hair.

A topical formulation according to the invention may also comprise a dermatologically, cosmetic or pharmaceutically acceptable carrier, diluent or excipients in which the metal oxide coated active agent particulates may be e.g., dispersed or suspended. The coated active agent may be easily dispersed or suspended in such a carrier, diluent or excipients, by for example mixing to achieve an effective dispersion or suspension. If necessary, high shear forces may be applied to facilitate fast and efficient mixing of the coated particles in the carrier.

In other embodiments, the carrier of a component of a system of the invention is in the form of a gel, a cream, a lotion, a cleanser, a saturated pad, an ointment, an oil, an emulsion, a paste, a milk, an aerosol, a powder, a foam, a wash (such as for example a wash-off or a wash-on) etc. In other embodiments, the carrier of a component of a system of the invention is an oil-in-water cream. In still further embodiments, the dispersing phase (i.e. the carrier of a component of a system of the invention) is aqueous based and comprises water as a dispersing medium.

It should be noted that each one of the components of a system of the invention may comprise a further at least one additive, such as but not limited to: a humectant (such as for example propylene glycol, glycerin, butylen glycol or polyethylene glycol), a buffer (such as for example citric acid aqueous solution, ammonium hydroxide solution phosphate buffer, borate buffer or carbonate buffer), a lubricant (such as for example cyclomethicone, dimethycone, castor oil, Iso propyl miristate, caprylic/capric triglyceride or octyl octanoate), an emulsifier (such as for example cetyl alcohol, glyceryl stearate, PEG-75 stearate, Ceteth-20, Steareth-20, Bis-PEG/PPG-16/16 PEG/PPG-16/16 dimethicone, sorbitan mono-oleate or alkyl poly glucoside), a moisturize (such as for example sodium PCA, sodium hyaluronate, panthenol or sodium latate), a soothing agent (such as for example natural herbal extracts such as Anthemis Nobilis flower extract, Chamomilla Recutita, Hamamelis Virginiana, burdock root, Argireline, Arnica Montana Extract, Shea Butter or aloe vera), a perfume, an exfoliating agent (such as for example polyethylene, glycolic acid 70%), a filler, an anti-irritating agent (such as for example allantoin), a chelating agent (such as for example EDTA), a preservative (such as for example imidazolidinyl urea, potassium sorbate, phenoxyethanol, methyl paraben, propyl paraben or benzyl alcohol), a detergent (such as for example polysorbate 20, sodium dodecyl sulfate or ceterimonium chlorid), a coloring agent, an antimicrobial agent (such as for example SD alcohol 40 or Chlorhexidine gluconate), a thickening agent (such as for example xanthan gum, guar gum, carboxy methyl cellulose, Carbomer or ethyl cellulose) and any combinations thereof.

In other embodiments of the invention, a system of the invention is suitable for topical administration onto at least a portion of the surface of the skin of a subject, wherein each component of said system is adaptable for concomitant, sequential, separate or parallel use.

It should be noted that each component of a system or a kit of the invention may be formed in a manner suitable for any particular condition of a skin area or mucosal membrane to be treated by way of selecting its ingredients, their concentration, the type of formulation and its consistency, the mode and the timing of use based on the condition of the subject, the area of the skin to be treated, the existing of a different treatment regimen, and other factors as known to a medical practitioner.

The term “subject” in the context of the present invention includes human and non-human mammals.

The term “surface of the skin” is meant to encompass any portion of the exposed or covered area, e.g., by hair, clothing, etc, of the skin or mucosal membrane, as disclosed hereinabove.

The term “topical administration” as used herein is meant to encompass local administration of a component of a system or a kit of the invention onto the surface of a skin or mucosal tissue of a subject without inducing any systemic effect.

In this context it should be noted that the components of a system of the invention may be administered according to any treatment profile. For example, each component may be administered concomitantly, sequentially, separately or parallel to the administration of any other component of a system of a kit of the invention. Each component may be administered adjacent to any other skin treatment procedure. Each component of a system of a kit of the invention may be administered independently either once or a few times a day (for example twice, three to ten times a day).

In some embodiments, a system of the invention is a three component system comprising: two components comprising metal oxide coated BPO, in the same or different concentrations and/or formulations, (components A and C) and another component which is free of metal oxide coated BPO (component B).

In other embodiments, each of the three components of said system is administered in the following regimens:
Component A, followed by Component B followed by Component C, (twice a day).
Component B, followed by Component A followed by Component C, (twice a day).
Component C, followed by Component B followed by Component A (twice a day).
Component A, followed by Component C followed by Component B (twice a day).
Component C, followed by Component A followed by Component B, (twice a day).
Component B, followed by Component C followed by Component A, (twice a day).

In other embodiments, a system of the invention is suitable for use in topical treatment of a skin condition.

In another aspect of the invention there is provided a method for treating, improving skin appearance of a subject or preventing a disease or condition of the skin or of a mucosal membrane, comprising topically administering onto said skin or mucosa a system of the invention.

In yet another aspect of the invention there is provided a method for treating skin lesions comprising topically administering onto said skin or mucosal membrane a system of the invention.

In another aspect the invention provides a method for treating skin comedones comprising topically administering onto said skin or mucosa a system of the invention.

In a further aspect the invention provides a method of treating acne comprising topically administering onto said skin or mucosal membrane a system of the invention.

The term “treating” or “treatment” as used herein includes any treatment of a condition or disease or disorder associated with a patient's skin or mucosal membrane, and includes change or improvement of its condition, inhibiting the disease or disorder (i.e. arresting its development), relieving at least one symptom associated with the disease or disorder (i.e. causing regression of the disease or disorder), and/or relieving a condition caused by the disease or disorder (i.e. symptoms of the disease).

Although individual needs may vary, determination of optimal ranges for effective amounts of the compositions is within the skill of the art. Generally, the dosage required to provide an effective amount of the active agent in the system, which can be adjusted by one skilled in the art, will vary depending on the age, health, physical condition, weight, type and extent of the disease or disorder of the recipient, frequency of treatment, the nature of concurrent therapy (if any) and the nature and scope of the desired effect(s).

The term “improving appearance” is meant to encompass a visible improvement of the condition of a skin area or mucosal membrane treated by a system or a kit of the invention. The improvement may appear as any change in the skin or mucosal membrane color, smoothness, uniformity, degree, intensity and number of lesions or wounds on the subject's skin or membrane area which may be due to any kind of skin condition or disorder such as in some embodiments bacterial, viral or fungal infection etc.

In some embodiments, the skin condition is a disease or disorder selected from acne, infection, inflammation, pruritis, psoriasis, seborrhea, contact dermatitis, skin cancers, skin photoaging, pigmentation, redness, rosacea, and any combination thereof.

In other embodiments of the invention, the components of a system of the invention are administered in a pre-determined administration schedule.

In another aspect of the invention there is provided a use of a system of the invention, for topical administration on the skin or mucosal membrane.

In some embodiments said topical administration is for treating a disease or disorder selected from acne, pruritis, psoriasis, seborrhea, contact dermatitis, infection, cancer, skin photoaging, pigmentation, redness, rosacea, inflammation, and any combination thereof. In some other embodiments said topical administration is for the treatment of skin lesions. In some further embodiments said topical administration is for the treatment of skin comedones. In yet further embodiments said topical administration is for the treatment of acne.

In yet a further aspect of the invention there is provided a kit comprising:

at least one component comprising at least one first active agent coated by metal oxide; and at least one skin treating component free of said active agent; and instructions for use.

In some embodiments a kit of the invention comprises at least one further component, each of said further components comprising at least one further active agent coated by metal oxide.

In other embodiments a kit of the invention is for use in the treatment or prevention of a disease or disorder selected from one or more of acne, rosecea, psoriasis, pruritis, skin photoaging, pigmentation, dermatitis, inflammation, mucosal infected areas, skin cancer, redness or any combination thereof. In other embodiments a kit of the invention is for use in the treatment or prevention of skin lesions. In yet other embodiments a kit of the invention is for use in the treatment or prevention of skin comedones. In further embodiments a kit of the invention is for use in the treatment or prevention of acne.

### BRIEF DESCRIPTION OF THE DRAWINGS

In order to understand the invention and to see how it may be carried out in practice, embodiments will now be described, by way of non-limiting example only, with reference to the accompanying drawings, in which:

FIG. 1 is a graphical representation of the tolerability (in %) parameters during use of Kits A to D within 4 weeks.

FIG. 2A-2C is a graphical representation of overall skin irritation rating of Kits A-D within 4 weeks of use.

FIG. 3 is a graphical representation of the change in total number of lesions during use of Kits A-D within 4 weeks.

FIG. 4 is a graphical representation of the % improvement of total number of lesions during use of Kits A-D within 4 weeks.

FIG. 5 is a graphical representation of the change in total number of non-inflated lesions during use of Kits A-D within 4 weeks.

FIG. 6 is a graphical representation of the % improvement of total number of non-inflated lesions during use of Kits A-D within 4 weeks

FIG. 7 is a graphical representation of % improvement in total number of black comedones during use of Kits A-D within 4 weeks.

FIG. 8 is a graphical representation of % improvement in total number of white comedones during use of Kits A-D within 4 weeks.

FIG. 9 is a graphical representation of the change in total number of inflamed lesions during use of Kits A-D within 4 weeks.

FIG. 10 is a graphical representation of the % improvement in total number of inflamed lesions during use of Kits A-D within 4 weeks.

FIG. 11 is a graphical representation of the overall product comfort score of each of Kits A to D within 1 and 4 weeks use.

### DETAILED DESCRIPTION OF EMBODIMENTS

According to one aspect of the invention there is provided a multi component topical system comprising: at least one component comprising at least one first active agent coated by metal oxide; and at least one skin treating component free of said active agent.

In some embodiments said metal oxide coating of said active agent is obtained by a process comprising:

(a) contacting a water-insoluble active agent with an ionic additive in an aqueous medium to obtain a dispersion of active agent particulates having charged surface;

(b) precipitating a metal oxide salt onto the surface of said active agent particulates to form a metal oxide coat on said particulates;

(c) optionally repeating step (b); and

(d) aging said metal oxide coating on said particulates.

The purpose of step (a) is to modify the electrical charge of the particulate active agent by using ionic additives such that the particulates are made reactive to the attachment of the metal oxide coating. In some embodiments, said ionic additive is selected from a cationic additive, an anionic additive, and a combination thereof.

As used herein the term “water-insoluble active agent” refers to a solid active agent having solubility in water of less than 1% w/w, typically less than 0.5% and at times less than 0.1% w/w at room temperature (20?C).

The “active agent particulates” constitutes the “core” of the particles obtained by a metal oxide coating process. The solid, water-insoluble active agent particulates are in a state of subdivision that allows suspension in water, e.g. in the form of a finely-divided powder, in some embodiments in the range of 0.3-100 micron. Such a particulate matter can readily be suspended in an aqueous system by stirring with or without the aid of a surfactant. The active agent particulates may be comprised of the active ingredient per se or may be comprised of the active ingredient and excipients (e.g. solid carrier). The terms “active agent particulates” and “particulate matter” are used herein interchangeably.

The term “dispersion” as used herein in step (a) of a metal oxide coating process refers to a solid dispersion of the particulate active agent in the aqueous medium.

Step (a) of the process may further comprise reducing the particle size of the particulate matter to the desired particle size, for example, by milling or homogenization.

The core (i.e. solid, water insoluble particulate matter) may be of any shape, for example, rod-like, plate-like, ellipsoidal, cubic, or spherical shape.

It is appreciated that some of the active agent particulates obtained by a metal oxide coating process as disclosed herein may at times be formed from two or more original solid water insoluble active agent particles and may accordingly include at times more than one core, such cores being separated from each other by a metal oxide region.

The weight of the solid, water-insoluble active agent particulates (core material) based on the total weight of the particle may be in the range of between about 99% to about 50% w/w. In some embodiments the total weight of the particle may be in the range of between about 97% to about 50% w/w.

In some embodiments, process step (b) is repeated between 4 and about 1,000 times. In a further embodiment the process for metal oxide coating of at least one active agent comprises repeating step (b) 4 to about 300 times. In yet a further embodiment step (b) is repeated between about 4 to about 100 times. In another embodiment step (b) is repeated between about 5 to about 80 times. In yet another embodiment step (b) is repeated between about 5 to about 50 times.

By the term “repeated between 4 and about 1,000 times” is meant that the process may be repeated 4, 5, 6, 7, 8, 9 . . . , etc. times up to and including about 1000 times.

It should be noted that during the metal oxide coating process of at least one active agent in a system of the invention, at least 50% of the content of the active agent particulates in the aqueous medium is in a solid state during the coating process.

In some embodiments the concentration of the ionic additives in the dispersion can be between about 0.001% to about 30%. In another embodiment the concentration of the ionic additives in the dispersion can be between about 0.01% to about 10% w/w. In a further embodiment the concentration of the ionic additives in the dispersion can be between about 0.1% up to about 5% w/w. In another embodiment the solid content of the water dispersion can be between about 0.1% to about 80% w/w. In a further embodiment content of the water dispersion can be between about 1% to about 60% w/w. In yet a further embodiment content of the water dispersion can be between about 3% to about 50% w/w.

In some embodiments the ionic additive is selected from a cationic additive, an anionic additive, and a combination thereof. The cationic additive may be a cationic surfactant and/or cationic polymer. The anionic additive may be an anionic surfactant and/or anionic polymer.

Without wishing to be bound by theory, as step (a) of the process for the coating of the active agent particulates recites, the active agent particulates are contacted with an ionic additive, for example by mixing it with a solution of an ionic surfactant, e.g., a cationic surfactant and/or cationic polymer or an anionic surfactant and a cationic additive (e.g. cationic surfactant and/or cationic polymer) to enable absorbance of said surfactants on at least a portion of the surface of the particulate active agent. The cationic surfactant and/or the cationic polymer and optionally further the anionic surfactant (or anionic polymer) need to be used in sufficient amounts to provide on average a positive charge on the surface of the particulate matter.

In another embodiment said metal oxide coating of said active agent is obtained by a process comprising:

(a) contacting at least one water-insoluble active agent, with a cationic additive in an aqueous medium to obtain a dispersion of active agent particulates having positive charges on at least a portion of their surface;

(b) precipitating a metal oxide salt onto at least a portion of the surface of said active agent particulates to form a metal oxide coating on said particulates;
(b1) contacting (e.g., by way of mixing) the metal oxide coated active agent particulates with at least one additive selected from (i) a further cationic additive, and (ii) a non-ionic additive, thereby obtaining metal oxide coated active agent particulates having at least one additive on at least a portion of the particulates' surface;
(b2) precipitating a metal oxide salt onto at least a portion of the surface of said metal oxide coated active agent particulates having at least one additive on at least a portion of the particulates' surface to form a further metal oxide coating on said particulates;

(c) optionally repeating steps (b1) and (b2); and

(d) aging the metal oxide coating on said particulates.

In some embodiments, the cationic additive of step (a) is a cationic additive or a mixture of a cationic additive with an anionic additive. In such embodiments, said cationic additive and said further cationic additive are the same or different. In some other embodiments, the cationic additive is a cationic surfactant and the further cationic additive is a cationic polymer.

In some other embodiments, the cationic additive of step (a) may be replaced with an anionic additive or a mixture thereof with a cationic additive.

According to another embodiment, the first cationic additive is a cationic surfactant and the additive in step (b1) is a non-ionic additive (e.g. a non-ionic polymer).

In some embodiments the cationic surfactant is selected from monoalkylquaternary ammonium salts, dialkyl quaternary ammonium salts, and mixtures thereof. In another embodiment the monoalkylquaternary ammonium salts are selected from benzethonium chloride, benzalkonium chloride, cetyltrimethylammonium chloride (CTAC), cetyltrimethylammonium bromide (CTAB), lauryltrimethylammonium chloride, stearyltrimethylammonium chloride, cetylpyridinium chloride, and mixtures thereof. In a further embodiment the monoalkylquaternary ammonium salt is cetyltrimethylammonium chloride. In yet a further embodiment the dialkyl quaternary ammonium salt is distearyldimethylammonium chloride.

The ionic additive may be an anionic surfactant. In some embodiments the anionic surfactant is selected from alkyl benzene sulphonic acid and salt, alkyl ether carboxylic acid and salt, alkyl sulphosuccinamate, alkyl sulphossucinate, alpha olefin sulphonate, aromatic hydrocarbon sulphonic acid and salt, fatty alcohol ethoxy sulphate, fatty alcohol sulphate, phosphate ester, and mixtures thereof. In another embodiment the alkyl benzene sulphonic acid salt is sodium dodecyl benzene sulphonate, the fatty alcohol sulphate is sodium lauryl sulphate, the alkyl sulphossucinates is sodium dioctyl sulphossucinate, and mixtures thereof. The anionic surfactant may be mixtures of any of the above.

Additional anionic and cationic surfactants which can be used by the metal oxide coating described herein are described in: John A. Wenninger et al. (Editors) International Cosmetic Ingredient Dictionary and Handbook (Eighth Edition 2000), Vol. 2 pp. 1140-1147, published by The cosmetic, Toiletry, and Fragnance Association, incorporated herein by reference in its entirety.

In another embodiment said process for coating of said at least one water insoluble active agent, independent of the number of times steps (b) or (b1) or (b2) are repeated, further comprises, following step (d):

(e) separating the metal oxide coated particulates from the aqueous medium and optionally rinsing and re-dispersing the coated at least one active agent in an aqueous medium.

In some embodiments said separation of the coated at least one active agent is conducted by a method such as filtration, centrifugation, decantation, dialysis, or by evaporation of the aqueous medium.

Additionally according to an embodiment of the present invention, step (b) comprises adding a metal oxide salt to the aqueous medium; and optionally acidifying the aqueous medium.

In another embodiment said coating process step (b) further comprises acidifying the aqueous medium. In another embodiment said coating process step (b2) comprises acidifying the aqueous medium.

In a further embodiment, process step (b1) further comprises adjusting the pH of the dispersion obtained in (b) to a value higher than the isoelectric point of the metal oxide before adding a further cationic additive.

In some embodiments step (b1) further comprising adjusting the pH of the dispersion obtained in (b) to a value higher than the isoelectric point of the metal oxide before adding a further cationic additive. In a further embodiment the pH value of at least about 1 unit higher than the isoelectric point of the metal oxide, before adding a further cationic additive.

In another embodiment step (b1) further comprising adjusting the pH of the dispersion obtained in (b) to a value higher than the isoelectric point of the metal oxide before adding one or both of (i) a further cationic additive, and (ii) a non-ionic additive. In another embodiment the pH value of at least about 1 unit higher than the isoelectric point of the metal oxide, before adding one or both of (i) a further cationic additive, and (ii) a non-ionic additive.

For example, in case the metal oxide is silica (e.g. having an isoelectric point in the range 1.7-2.5) the pH may be at least in the range of about 2.5-6.5.

Without being bound by theory it should be noted that the adjustment of pH of the dispersion to a value higher than the isoelectric point of the metal oxide is conducted in order to form negatively charged metal oxide on the particulate surface to be bound to the positive charges of the further cationic additive thus enabling the attachment of the further cationic additive to the surface of the particulate matter.

In some embodiments the non-ionic additive is selected so it adheres to the surface (“surface-adherent”). An example is a non-ionic polymer. The non-ionic additive may be used alone or in addition to a further cationic surfactant. Without wishing to be bound by theory, the surface-adherent property may be through groups such as hydroxyl or amine groups permitting hydrogen bonding. This allows adhesion of a further coating of metal oxide on the previously precipitated metal oxide coating.

In a further embodiment the weight ratio between active agent particulates/metal oxide salt, in each of the steps (b) or (b2) is between about 5,000/1 to about 20/1. In another embodiment the ratio is between about 5,000/1 to about 30/1. In a further embodiment the ratio is between about 5,000/1 to about 40/1. In yet a further embodiment the ratio is between about 1,000/1 to about 40/1. In another embodiment the ratio is between about 500/1 to about 80/1.

In some embodiments the weight ratio of the ionic additive to the water-insoluble active agent particulates is in the range is between about 1:1000 to about 1:10. In another embodiment the ratio is in the range of between about 1:200 to about 1:50. In a further embodiment the ratio is about 1:100. The ratios indicated above refer to an ionic additive such as the first cationic additive or to the combination of a first cationic additive and an anionic additive. A further cationic additive may be a cationic polymer, a cationic surfactant, or mixtures thereof. The cationic surfactant may be as described above.

In another embodiment the ratio between active agent/cationic additive, in step (b1) is between about 25,000/1 to about 50/1. In another embodiment the ratio is between about 5,000/1 to about 100/1. hi yet a further embodiment the ratio is between about 2000/1 to about 200/1.

In another embodiment the weight ratio between active agent particulates/cationic additive in step (b1) is between about 10,000/1 to about 100/1. In another embodiment the ratio is between about 5000/1 to about 200/1.

In another embodiment the weight ratio of the first coated particulate matter (i.e. in step (b1)) to a further cationic additive is in the range of between about 25,000/1 to about 50/1. In another embodiment the ratio is in the range of between about 5,000/1 to about 100/1. In a further embodiment the ratio is in the range of between about 2000/1 to about 200/1.

In another embodiment the weight ratio of the further processed coated active agent particulates (e.g. in the repeated steps described in step (c)) to the further cationic additive is in the range of between about 25,000/1 to about 50/1. In another embodiment the ratio is between about 5,000/1 to about 100/1. In a further embodiment the ratio is in the range of between about 2000/1 to about 200/1.

In another embodiment the weight ratio of the further processed coated active agent particulates (e.g. in the repeated steps described in step (c)) to the further cationic additive is in the range of between about 10,000/1 to about 100/1. In another embodiment the ratio is in the range of between about 5000/1 to about 200/1.

In case a non-ionic additive (e.g. non-ionic polymer) is used alone or in addition to a further cationic additive, the weight ratios of the of the first coated particulate matter to the (i) non-ionic additive or (ii) a combination of a non-ionic additive and further cationic additive, and the weight ratios of the further processed coated particulate matter to the (i) non-ionic additive or (ii) the combination of the non-ionic additive and further cationic additive, may be as indicated above with respect to the further cationic additive.

The cationic polymer is selected from poly(ethyleneimine) (PEI), poly(dimethyldiallylammonium chloride) (PDAC), poly(acrylamide-co-diallyl-dimethylammonium chloride)(polyquaternium-7), poly(allylamine hydrochloride) (PAH), Chitosan, polylysine, and mixtures thereof.

In some embodiments a further cationic polymer may also be a copolymer of non-ionic and ionic monomers such as pyrrolidone/dimethylaminoethyl methacylate copolymer.

According to another embodiment of the present invention a further cationic additive is selected from colloidal alumina, colloidal ceria (CeO₂), colloidal alumina coated silica (such as Ludox CL, Sigma-Aldrich), and mixtures thereof.

The further cationic additive may be a colloidal metal oxide bearing a positive charge such as described above (e.g. colloidal alumina, colloidal ceria (CeO₂), colloidal alumina coated silica, or mixtures thereof).

The non-ionic additive used in the process is in some embodiments a non-ionic polymer. The non-ionic polymer may be for example polyvinylalcohol, polyvinylpyrrolidone, and mixtures thereof.

Further according to an embodiment of the present invention, the process further comprises drying the obtained coated active agent particulates.

Still further according to an embodiment of the present invention, the drying is by a method selected from spray drying, lyophilization, oven drying, vacuum drying, and fluidized bed.

After the aging of step (d) a strengthened and dense coating of metal oxide is obtained about the active agent particulates.

In another embodiment step (d) further comprises raising the pH to a value in the range 3-9 and mixing the suspension in this pH.

According to an embodiment of the metal oxide coating process step (d) comprises raising the pH to a value in the range of between about 3 to about 9. In a further embodiment the pH ranges between about 5 to about 7, and mixing, e.g. by stirring, the suspension (dispersion) in this pH range, e.g. for a period of at least 2 h (two hours). In another embodiment stirring is performed for about 2 to about 96 h. In another embodiment stirring is performed for about 2 to about 72 h. In yet a further embodiment stirring is performed for at least 10 h (for example between about 10 to about 72 h). In another embodiment the stirring is performed in the range of between about 200 to about 500 rpm.

In a further embodiment process step (d) further comprises raising the pH to a value in the range 3-9 and mixing the suspension in this pH for a period of at least 2 h.

The aging of the metal oxide coating of the at least one active agent particulates may be conducted at a temperature of between about 4 to about 90?C. In another embodiment the aging step is performed between about 15 to about 60?C. In another embodiment the aging is conducted at a temperature of between about 20?C to about 40?C.

Thus the repeated steps of coating and aging at the end of the process also enable the growth of thicker and stronger coating of metal oxide. In some embodiments aging is not conducted between the repeated coating steps (i.e. between the repeated coating step (b)), but only at the end of the coating process. Thus, in some embodiments the aging step is conducted only at the end of the process described herein.

In another embodiment of the invention said metal oxide salt is selected from sodium silicate, potassium silicate, sodium aluminate, potassium aluminate, sodium titanate, potassium titanate, sodium zirconate, potassium zirconate, and mixtures thereof.

According to certain embodiments, the process may further comprise adding a colloidal metal oxide suspension. In a further embodiment the colloidal metal oxide suspension is an aqueous-based suspension (comprising nanometric metal oxide (nanoparticles of metal oxide)) during the coating process (e.g. in step (b) in one or more of its repeated steps).

In another embodiment the colloidal metal oxide suspension is selected from colloidal silica suspension, colloidal titania suspension, colloidal alumina suspension, colloidal zirconia suspension, colloidal ZnO suspension, and any mixtures thereof.

In some embodiments the average particle size diameter of the nanometric metal oxide is in diameter is in the range between about 5 to about 100 nm. The weight ratio of the nanometric metal oxide to the metal oxide salt may be in the range of between about 95:5 to about 1:99. In another embodiment the ratio is in the range of between about 80:20 to about 5:95. In another embodiment the ratio is in the range of between about 70:30 to about 10:90. The ratio is in the range of between about 60:40 to about 20:80. The weight ratio of the nanometric metal oxide to the metal oxide salt may be about 50:50.

In another embodiment of the invention a process of metal oxide coating of said active agent particulates further comprises chemically modifying at least a portion of the outmost metal oxide coating of said active agent particulates. In a further embodiment said modification comprises attaching hydrophobic groups to at least a portion of the outmost metal oxide coating of active agent particulates. In yet a further embodiment of the invention chemical modification comprises reacting silanol groups on at least a portion of the outmost metal oxide coating of active agent particulates with precursors selected from monohalotrialkyl silane, dihalodialkyl silane, trihaloalkyl silane, monoalkoxytrialkyl silane, dialkoxydialkyl silane, trialkoxyalkyl silane, and mixtures thereof.

In some embodiments the alkyl group includes 1-18 carbon atoms. In another embodiment the alkyl group includes 1-6 carbon atoms. In another embodiment the alkyl is methyl. The alkyl groups may be substituted by one or more flouro atoms. In another embodiment the alkoxy group includes 1-6 carbon atoms. In a further embodiment the alkoxy group includes 1-2 carbon atoms.

The halo group may be for example chloro, bromo, iodo, fluoro. In some embodiments the halo groups are chloro and bromo. In another embodiment the aryl is phenyl or benzyl.

The precursors react with the silanol groups on the surface of the metal oxide coating to form a siloxane bond.

The attachment of the hydrophobic groups to the surface of the metal oxide coating can be performed by reacting the dried coated particulate with the above precursors. The procedure for attaching hydrophobic groups to the metal oxide can be conducted as follows: a dried powder of coated particulate is suspended in an organic solvent such as toluene. A precursor (hydrophobization reagent) from the list above such as dimethyldichlorosilane is added to the organic phase (mixture), optionally in the presence of a halogen scavenger such as trialkyl amine or triethanol amine. The organic mixture is refluxed for at least about 24 hours to obtain coverage of the metal oxide coating with the hydrophobic groups via attachment of the hydrophobic groups to the silanol groups on the surface of the metal oxide coating.

In a further embodiment of the present invention said metal oxide coating of the active agent has a width (thickness) of 0.1-10 micron. Further according to an embodiment of the present invention the obtained metal oxide coating has a width of about 0.1, 0.2, 0.3, 0.5, 0.7, 1, 1.5, 2 or 5 micron or above. In some embodiments the obtained metal oxide coating has a width of up to 10 micron.

The width of the metal oxide coating may be determined for example by a Transmission Electron Microscope or Confocal Microscope such that in a circular cross sectional area of the particulate the smallest width is at least e.g. 0.1 micron (the width is determined as the smallest distance from the surface of the particle (i.e. metal oxide surface) to the core-metal oxide interface).

According to an embodiment of the present invention the metal oxide coated active agent particulates have a diameter of about 0.5 to about 100 micron. In another embodiment the diameter of the particulates is in the range about 1 to about 50 micron. In yet a further embodiment the diameter of the particulates is in the range about 2 to about 30 micron.

It must be noted that, as used in this specification and the appended claims, the singular forms “a”, “an” and “the” include also plural referents unless the content clearly dictates otherwise.

Throughout this specification and the claims which follow, unless the context requires otherwise, the word “comprise”, and any lingual variations thereof, should be understood to imply the inclusion of a stated integer or step or group of integers or steps, but not the exclusion of any other integer or step or group of integers or steps.

The following Examples are representative of techniques employed by the inventors in carrying out aspects of the present invention. It should be appreciated that while these techniques are exemplary embodiments for the practice of the invention, those skilled in the art, in light of the present disclosure, will recognize that numerous modifications can be made without departing from the spirit and intended scope of the invention.

### EXAMPLES

In the examples below, all % values referring to a solution are in (w/w). All % values, referring to dispersions are in (w/w).

All solutions used in the examples below unless otherwise stated refer to an aqueous solution of the indicated ingredient.

### Example 1

### Silica Coating of BPO

Step 1: milling: 110 g. of hydrous BPO 75% (USP grade from Sigma) were suspended in 152 g. of 0.4% CTAC solution containing 0.001% silicon antifoam. The BPO was milled using a stator rotor mixer (Kinematika polytron 6100 operated at 15,000 rpm/25m/s). The milling was stopped when the particle size distribution (PSD) of the suspension was d(0.9)<35 □m or the temperature has reached 50 C. The final suspension was cooled to room temperature.

Step 2a: coating option #1: During the coating procedure the suspension was stirred with a mechanical dissolver, 80 mm, at 500 RPM at all times. The pH of the milled BPO suspension was corrected to 8 using NaOH 5N solution. A portion of 1 g of 15% sodium silicate solution (15% w/w as SiO₂) was added and the suspension was stirred for 5 min. A portion of 1 g of 3% Polyquaternium 7 was added and the suspension was stirred for 5 min. pH was adjusted to 6-7 using 5N HCl solution.

This procedure was repeated for 5-100 times in order to create a series of silica coatings around BPO having different thickness.

Step 2b: coating option #2: During the coating procedure the suspension was stirred with a mechanical dissolver, 80 mm, at 500 RPM at all times. The pH of the milled BPO suspension was corrected to 8 using NaOH 5N solution. A portion of 2.5 g of 15% sodium silicate solution (15% w/w as SiO₂) was added and the suspension was stirred for 5 min. A portion of 2.5 g of 3% Polyquaternium 7 was added and the suspension was stirred for 5 min. pH was adjusted to 6-7 using 5N HCl solution.

This procedure was repeated for 5-100 times in order to create a series of silica coatings around BPO having different thickness.

The aging step: The coated BPO suspension at pH 6.5 was kept for aging at room temperature (25 C+/−2) under gentle agitation for 24 hrs.

### Example 2

### Silica Coating of BPO Using Anionic Surfactant

Step 1: milling: 110 g. of hydrous BPO 75% (USP grade from Sigma) were suspended in 152 g. of 0.4% sodium dodecyl sulphonate (SDS) solution containing 0.005% silicon antifoam. The BPO was milled using a stator rotor mixer (Kinematika polytron 6100 operated at 15,000 rpm/25 m/s). The milling was stopped when the particle size distribution (PSD) of the suspension was d(0.9)<35 □m or the temperature has reached 50 C. The final suspension was cooled to room temperature and a portion of 1-2.5 g of 3% Polyquaternium 7 was added and the suspension was stirred for 5 min.

Step 2a: coating option #1: During the coating procedure the suspension was stirred with a mechanical dissolver, 80 mm, at 500 RPM at all times. The pH of the milled BPO suspension was corrected to 8 using NaOH 5N solution. A portion of 1 g of 15% sodium silicate solution (15% w/w as SiO₂) was added and the suspension was stirred for 5 min. A portion of 1 g of 3% Polyquaternium 7 was added and the suspension was stirred for 5 min. pH was adjusted to 6-7 using 5N HCl solution.

This procedure was repeated for 5-100 times in order to create a series of silica coatings around BPO having different thickness.

Step 2b: coating option #2: During the coating procedure the suspension was stirred with a mechanical dissolver, 80 mm, at 500 RPM at all times. The pH of the milled BPO suspension was corrected to 8 using NaOH 5N solution. A portion of 2.5 g of 15% sodium silicate solution (15% w/w as SiO₂) was added and the suspension was stirred for 5 min. A portion of 2.5 g of 3% Polyquaternium 7 was added and the suspension was stirred for 5 min. pH was adjusted to 6-7 using 5N HCl solution.

This procedure was repeated for 5-100 times in order to create a series of silica coatings around BPO having different thickness.

The aging step: The coated BPO suspension at pH 6.5 was kept for aging at room temperature (25C+/−2) under gentle agitation for 24 hrs.

### Example 3

### Topical Kits Comprising Metal Oxide Coated BPO

BPO with metal oxide coating (15% in suspension)

| No. | Ingredients | Function | % |
|---|---|---|---|
| 1 | Hydrous Benzoyl Peroxide | Active Agent | 3.00-50.00 |
| 2 | Sodium Silicate | Silica precursor | 0.10-10.00 |
| 3 | Cetrimonium Chloride | Stabilizing surfactant | 0.01-1.00 |
| 4 | Polyquaternium 7 | Stabilizing polymer | 0.01-5.00 |
| 5 | Lactic Acid | pH modifier | 0.01-1.00 |
| 6 | Citric Acid | pH modifier | 0.01-3.00 |
| 7 | Hydrochloric Acid | pH modifier | 0.01-1.00 |
| 8 | Purified Water | Suspending solvent | Up to 100 |

The metal oxide coating process was performed in accordance with process steps as disclosed in Example 1 above.

Kit A:

Component A1 (cleanser)

| No. | Ingredients | Function | % |
|---|---|---|---|
| 1 | Water purified | Filler | Up to 100 |
| 2 | Propylene Glycol | Humectant, Cleansing | 0.10-15.00 |
| | | agent | |
| 3 | Glycerin | Humectant | 0.10-15.00 |
| 4 | Citric Acid | Buffer | 0.01-1.00 |
| 5 | Cyclomethicone | Lubricant, Cleansing | 0.10-20.00 |
| | | agent | |
| 6 | Cetyl Alcohol, Glyceryl | Emulsifier | 0.10-5.00 |
| | Stearate, PEG-75 Stearate, | | |
| | Ceteth-20, Steareth-20 | | |
| 7 | Bis-PEG/PPG-16/16 PEG/ | Emulsifier | 0.10-10.00 |
| | PPG-16/16 Dimethicone, | | |
| | Caprylic/Capric | | |
| | Triglyceride | | |
| 8 | Sodium PCA | Moisturizer | 0.01-1.00 |
| 9 | Sodium Hyaluronate | Moisturizer | 0.01-1.00 |
| 10 | Propylene Glycol, Water, | Tonic, Soothing and | 0.01-2.00 |
| | Anthemis Nobilis Flower | Calming Agent | |
| | Extract | | |
| 11 | Fragrance | Perfume | 0.001-0.50 |
| 12 | Metal oxide coated BPO, | Coated Active Agent | 2.00-60.00 |
| | 15% suspension | | |
| 13 | Polyethylene | Exfoliant agent | 0.10-10.00 |

Component A3 (lotion)

| No. | Ingredients | Function | % |
|---|---|---|---|
| 1 | Cyclomethicone | Lubricant | 0.10-20.00 |
| 2 | Cetyl Alcohol, Glyceryl | Emulsifier | 0.10-10.00 |
| | Stearate, PEG-75 | | |
| | Stearate, Ceteth-20, | | |
| | Steareth-20 | | |
| 3 | Purified water | Filler | Up to 100 |
| 4 | Allantoin | Moisturizing, Anti | 0.01-1.00 |
| | | irritating agent | |
| 5 | Panthenol | Moisturizing agent | 0.10-5.00 |
| 6 | Xanthan Gum | Thickening agent | 0.01-3.00 |
| 7 | Glycerin | Humectant | 0.5-30.00 |
| 8 | Fragrance | Perfume | 0.01-1.00 |
| 9 | Metal oxide coated BPO, | Coated Active Agent | 2.00-60.00 |
| | 15% suspension | | |
| 10 | Citric Acid | Buffer | 0.01-1.00 |

Component A2 (toner)

| No. | Ingredients | Function | % |
|---|---|---|---|
| 1 | Water | Filler | Up to 100 |
| 2 | EDTA | Chelating agent | 0.01-0.50 |
| 3 | Imidazolidinyl Urea | Preservative | 0.01-1.00 |
| 4 | Potassium Sorbate | Preservative | 0.01-1.00 |
| 5 | Propylene Glycol | Humectant/ | 0.10-20.00 |
| | | Solvent | |
| 6 | Glycerin | Humectant | 0.10-20.00 |
| 7 | Polysorbate 20 | Detergent | 0.10-5.00 |
| 8 | Glycolic Acid | Exfoliant | 0.10-20.00 |
| 9 | Ammonium Hydroxide | Buffer | 0.01-1.00 |
| 10 | Water, Butylene Glycol, | Astringent | 0.01-2.00 |
| | Hamamelis Virginiana (Witch | | |
| | Hazel) Leaf Extract | | |
| 11 | Propylene glycol, Water, | Calming agent | 0.01-2.00 |
| | Anthemis nobilis flower extract | | |
| 12 | Aloe barbadensis leaf extract | Calming agent | 0.01-5.00 |
| 13 | Sodium PCA | Moisturizer/ | 0.01-5.00 |
| | | Humectant | |
| 14 | Panthenol | Moisturizer | 0.01-2.00 |
| 15 | Allantoin | Anti irritant | 0.01-2.00 |
| 16 | Yellow 5 | Coloring Agent | 0.001-0.100 |
| 17 | Blue 1 | Coloring Agent | 0.001-0.1000 |

Kit B:

Component B1 (cleanser)

| No. | Ingredients | Function | % |
|---|---|---|---|
| 1 | Water purified | Filer | Up to 100 |
| 2 | Propylene Glycol | Humectant, | 0.10-20.00 |
| | | Cleansing agent | |
| 3 | Glycerin | Humectant | 0.10-20.00 |
| 4 | Citric Acid | Buffer | 0.10-1.00 |
| 5 | Cyclomethicone | Lubricant, | 0.10-20.00 |
| | | Cleansing agent | |
| 6 | Cetyl Alcohol, Glyceryl | Emulsifier | 0.10-10.00 |
| | Stearate, PEG-75 Stearate, | | |
| | Ceteth-20, Steareth-20 | | |
| 7 | Bis-PEG/PPG-16/16 PEG/ | Emulsifier | 0.10-10.00 |
| | PPG-16/16 Dimethicone. | | |
| | Caprylic/Capric Triglyceride | | |
| 8 | Sodium PCA | Moisturizer | 0.01-1.00 |
| 9 | Sodium Hyaluronate | Moisturizer | 0.01-1.00 |
| 10 | Chamomilla Recutita (Matricaria) | Tonic, Soothing | 0.01-2.00 |
| | Flower Extract | Calming Agent | |
| 11 | Fragrance (clear day) | Perfume | 0.01-1.00 |
| 12 | Metal oxide coated BPO, | Active ingredient | 2.00-60.00 |
| | 15% suspension | | |
| 13 | Polyethylene | Exfoliant agent | 0.10-10.00 |

Component B2 (toner)

| No. | Ingredients | Function | % |
|---|---|---|---|
| 1 | Water | Filler | Up to 100 |
| 2 | EDTA | Chelating agent | 0.01-1.00 |
| 3 | Imidazolidinyl Urea | Preservative | 0.01-1.00 |
| 4 | Potassium Sorbate | Preservative | 0.01-1.00 |
| 5 | Propylene Glycol | Humectant/Solvent | 0.10-20.00 |
| 6 | Glycerin | Humectant | 0.10-20.00 |
| 7 | Polysorbate 20 | Detergent | 0.10-5.00 |
| 8 | Glycolic Acid | Exfoliant | 0.10-20.00 |
| 9 | Ammonium Hydroxide | Buffer | 0.01-2.00 |
| 10 | Salicylic Acid | Active Ingredient | 0.5-10.00 |
| 11 | SD Alcohol 40 | Antimicrobial agent | 1.00-50.00 |
| 12 | Allantoin | Anti irritant agent | 0.01-1.00 |

Component B3 (lotion)

| No. | Ingredients | Function | % |
|---|---|---|---|
| 1 | Cyclomethicone | Lubricant | 0.10-20.00 |
| 2 | Cetyl Alcohol, Glyceryl | Emulsifier | 0.10-10.00 |
| | Stearate, PEG-75 | | |
| | Stearate, Ceteth-20, | | |
| | Steareth-20 | | |
| 3 | Furified water | Filler | Up to 100 |
| 4 | Allantoin | Moisturizing & | 0.01-1.00 |
| | | Anti irritating agent | |
| 5 | Panthenol | Moisturizing agent | 0.01-5.00 |
| 6 | Xanthan Gum | Thickening agent | 0.01-3.00 |
| 7 | Glycerin | Humectant | 0.10-20.00 |
| 8 | Fragrance (Perfect Skin) | Perfume | 0.01-1.00 |
| 9 | Metal oxide coated BPO, 15% | Coated Active | 2.00-60.00 |
| | suspension | Agent | |
| 10 | Citric Acid | Buffer | 0.01-2.00 |

### Example 4

### Topical Kits Comprising Metal Oxide Coated BPO

A double blind randomized study was planned to include 80 patients (20 in each treatment group). Patients were randomly allocated to receive either kit A, B, C or D, as listed below.

### Test Kits

Four kits were compared in this study:

Kit A (of the invention):

Component A1 (Cleanser): 4% silica coated BPO as active ingredient, botanicals with calming/soothing properties and exfoliating beads

Component A2 (Toner): Glycolic acid with calming and soothing botanicals

Component A3 (Lotion): 4% silica coated BPO as active ingredient, and botanicals with improved efficacy properties

Kit B (of the invention):

Component B1 (Cleanser): 7% silica coated BPO as active ingredient and botanicals with calming/soothing properties and exfoliating beads.

Component B2 (Toner): 2% salicylic acid, with calming and soothing properties agent.

Component B3 (Lotion): 7% silica coated BPO as active ingredient and botanicals with improved efficacy properties.

Kit C (control):

Component C1 (Cleanser): free uncoated 2.5% BPO as active ingredient, botanical extracts.

Component C2 (Toner): Glycolic acid, botanical extracts.

Component C3 (Lotion): free uncoated 2.5% BPO as active ingredient, botanicals

Kit D (control):

Component D1 (Cleanser): free uncoated 7% BPO as active ingredient, botanical extracts

Component D2 (Toner): 2% salicylic acid as active ingredient, botanical extracts

Component D3 (Lotion): free uncoated 7% BPO as active ingredient. Inactive ingredients: botanicals

### Treatment Regiment

The patients were requested to apply the kits' components for one month twice a day, morning and evening sequentially as follows:

Step 1—Cleanser Component

Apply a small amount to dampened skin and massage gently. Rinse thoroughly with warm water and pat dry. Follow with Step 2—Toner component.

Step 2—Toner Component

Apply to cleansed skin with cotton ball or pad. Allow toner to dry and follow with Step 3—Lotion component.

Step 3—Lotion Component

On clean skin apply a thin layer to the entire affected area. Allow drying. Do not rinse.

Patients were evaluated by the study Principle Investigator for efficacy and safety parameters on days 0, 8, 15, 22 and 29 (±3 days). The total duration of the study was 4 weeks. The first subject was enrolled on April 2008 and the last subject returned to the clinic on June 2008.

### Selection of Study Population

### Inclusion Criteria

To be considered eligible to participate in this study, each subject had to meet the inclusion criteria listed below:
1. Patient had mild to moderate facial acne vulgaris as determined by:
   At least 3 open and/or closed comedones (non-inflammatory lesions)
   At least 6 papules and/or pustules (inflammatory lesions)
   No significant nodulocystic acne (? 4 lesions)
   No more than 25 lesions for each type (non-inflammatory plus inflammatory lesions)
2. Males and Females between 16 and 35 years of age.
3. Female subjects used effective method of contraception and when using contraceptive pills they should have not make any changes within 3 months prior to the enrollment to the study.
4. Subjects were willing to refrain from using any topical (facial) products or medicated shampoos for the entire duration of the study.
5. Subjects provided written Informed Consent Form (if subject was 16-18 years old, the Informed Consent Form had to be signed by the legal representative of the subject).
6. Subjects were willing to use the full 3 steps kit twice a day throughout the study duration.

### Exclusion Criteria

Subjects were to be excluded from the study if one of the following criteria was met:
1. Known hyper-sensitivity to Benzoyl Peroxide or Salicylic Acid/Glycolic Acid.
2. Medical or psychiatric conditions that affected the subject's ability to give informed consent, or complete the study.
3. Pregnant or lactating females
4. Use of medications which may have influenced skin surface such as retinoids, antibiotics and corticosteroids within the last 4 weeks prior to enrollment and use of Isotretinoins within the last 6 months (only one treatment course has been allowed prior to the study), topical anti-acne medications (during the 2 weeks prior to the trial), change in contraceptive pills 3 months prior to enrollment and during the course of the study.
5. Other known diseases, including severe allergy, chronic liver or kidney disease, malignancy or contagious diseases, such as HIV or hepatitis
6. Subjects who underwent cosmetic or dermatological procedures during one month prior to the study for treating acne, acne scarring or hyperpigmentation, laser treatments, microdermabrasion, light treatment etc. or use of artificial sun bath or having a sun holiday during 2 weeks prior to enrollment.
7. Use of comedogenic cosmetics.
8. Alcohol or drug abuse, according to assessment by the investigator
9. Participation in a clinical trial within 30 days prior to the study.

### Schedule of Assessments and Study Procedures

### Screening Visit

During the screening visit each subject was evaluated for the inclusion and exclusion criteria. Eligible subjects signed the informed consent form and received a subject number (subsequent numbers) identical to the number on the treatment kit. The medical history and history of acne were reviewed including any medication taken within the last 30 days prior to the study. Demography details were collected. Acne severity was evaluated by the Investigator and all lesions were counted. All female subjects underwent urine pregnancy test.

### Baseline Visit

During the baseline visit subjects were interviewed to establish whether any change occurred in eligibility criteria since the previous visit, including any use of concomitant medication. The Investigator counted all the lesions and photographs were taken for documentation. Each subject received the appropriate medication kit, a diary to complete at home and instructions for use. Subjects were instructed to return to the clinic after 1 week.

Visits 3, 4 and 5 (Days 8, 15 and 22±3 days)

During the weekly visits subjects were examined and efficacy and safety parameters were evaluated by the Investigator.

Efficacy was assessed by counting lesions and the level of improvement was determined by the Investigator. Photographs of the face were taken at each visit.

Safety was assessed by the Investigator by reviewing and recording tolerability parameters, all adverse events and concomitant medications.

A questionnaire was completed by the patients during visit 3.

End of Study Visit (Visit 6, Day 29±3 days)

During this visit subjects were examined and efficacy and safety parameters were evaluated.

The Investigator counted the lesions and photographs of the face were taken. Subjects have completed a second questionnaire during the visit. Adverse events and concomitant medication were reviewed and recorded.

### Concomitant Medication

All concomitant medications taken by subjects during the study, including topical facial treatments, had to be documented.

Subjects were instructed to use non—comedogenic cosmetics. During the study, subjects were not allowed to use Retinoids of any kind, corticosteroids of any kind, Antibiotics of any kind and any anti-acne medication.

### Tolerability and Safety Assessment

At each visit the subjects were evaluated visually by the Investigator for any dermal irritation (erythema, edema, dryness, scaling/peeling) and subjective tolerance (stinging/burning, itching, tightness)

Each end-point was graded from 0 to 3 as follows:

0=No visible abnormal reaction.

0.5=Very slight

1=Slight

2=Moderate

3=Severe

In addition, subjects were questioned by the Investigator regarding any other adverse events, which might have occurred during the preceding week.

Adverse events were recorded at all visits.

The relation to the study medication was defined as: unrelated, possible, probable and likely.

Anticipated adverse events (mild dryness, peeling or stinging/burning sensations) were reported as adverse events only when un-tolerated by the subjects.

### Efficacy Assessment

Lesions from the entire facial area were counted and assessed during each visit. Lesions were categorized as non-inflamed and inflamed.

The non-inflamed lesions included blackheads and whiteheads—comedones.

The inflamed lesions included papules, pustules, nodules and cysts.

The Investigator was requested to assess the improvement of the acne condition by using the Global Assessment (IGA), grading improvement as:

0=Clear skin with no inflammatory or non-inflammatory lesions.

1=Almost clear, rare non-inflammatory lesions with no more than one small inflammatory lesion.

2=Mild severity greater than grade 1, some non-inflammatory lesions with no more than few inflammatory lesions (papules/pustules only, no nodular lesions)

3=Moderate severity, greater than grade 2, up to many non-inflammatory lesions and may have some inflammatory lesions, but no more than one small nodular lesion.

4=Severe; greater than grade 3, up to many inflammatory and non-inflammatory lesions, but no more than a few nodular lesions.

Subjects were also requested to complete a questionnaire in order to evaluate their level of satisfaction from the treatment. The questionnaire was to be completed during visit 3 and visit 6.

### Treatment Compliance

During each visit subjects were requested to report about missed treatments by any of the 3 kit's compounds. Subjects were considered as non-compliant if they did not apply the medication for more than 10% of the time on subsequent days.

### Statistical Analysis

### Sample Size

A total of 80 subjects (20 in each group) were to be enrolled to the study in order to perform statistical analysis on at least 64 subjects (taking in account drop-out rate of 20%). This number of subjects was not planned to achieve statistically significant results.

### Statistical Plan

Differences between four groups of continuous variables were analyzed by ANOVA model with post hoc Scheffe & Gabriel tests and Brown-Forsythe Robust Tests when appropriate.

Differences between four groups of ordinal variables were analyzed by Kruskall-Wallis test.

Pair-wise comparisons were analyzed using Mann-Whitney test with Bonferroni correction for multiple comparisons.

Changes from baseline of continuous variables were analyzed by repeated measures model with post-hoc tests and contrasts

Categorical variables were tested using □2 test or Fischer exact test.

All tests were two-sided.

The statistical analysis was based on per-protocol set.

Compliance was analyzed both based on intent to treat (ITT) and per protocol data sets.

No imputation of missing values was made.

The significance level was set at ?=0.05 (0.95 confidence intervals), p values were presented for all statistical tests.

No adjustment of p values was done for multiplicity of measurements.

Multiple comparisons were tested by Shceffe, Dunnett, tests or Bonferroni correction.

### Criteria for Evaluating Subjects

The intent to treat (ITT) group included all the subjects that received at least one treatment. The per-protocol (PP) population, valid for efficacy assessment was defined as all evaluable subjects who completed the 4 weeks treatment without having any major protocol violations.

### Study Results

### Disposition of Subjects

78 subjects were enrolled into the study, 20 assigned to Kit (A), 19 assigned to kit (B), 20 assigned to Kit (C), and 19 were assigned to kit (D). The per-protocol group included 67 subjects. Eleven subjects were excluded from the per-protocol analysis: 1 subject in Kit (A) was excluded due to an adverse event. 3 subjects in kit (B) were excluded, 2 due to use of prohibited medication and one has lost to follow up. 2 subjects in Kit (C) were excluded, 1 due to an adverse event and 1 has lost to follow up. 5 patients withdrew from kit (D) due to withdrawal of consent and or have lost to follow-up.

### Baseline Characteristics

The four treatment groups were similar in their baseline characteristics (demographics, duration of acne condition and severity of baseline condition). 78.9%, 81.3%, 83.3% and 64.3% of the subjects in the A, B, C and D treatment group (respectively) were females. The mean age was 18.5±4 years (min 15.8 max 31 years) in group A, 19.1±2.9 years (min 16 max 27 years) in group B, 18.7±2.4 years (min 16 max 24 years) in group C and 17.1±1.3 years (min 16 max 20 years) group D.

TABLE 1
Acne Condition at Baseline

| | Group | | Group | | Group | | Group | | |
|---|---|---|---|---|---|---|---|---|---|
| | A | | B | | C | | D | | |
| | Mean | SD | Mean | SD | Mean | SD | Mean | SD | P value |
| Blackheads | 5.0 | 2.3 | 6.3 | 4.3 | 5.1 | 2.8 | 6.9 | 2.9 | 0.218 |
| comedos | | | | | | | | | |
| Whiteheads | 13.7 | 5.5 | 11.8 | 5.0 | 13.6 | 5.4 | 14.6 | 2.8 | 0.299 |
| comedos | | | | | | | | | |
| Papules | 10.4 | 3.9 | 10.6 | 3.6 | 11.8 | 3.8 | 10.6 | 4.6 | 0.688 |
| Pustules | 2.0 | 1.7 | 1.4 | 1.5 | 1.8 | 1.9 | 1.6 | 1.2 | 0.686 |
| Nodules | 0.0 | 0.0 | 0.0 | 0.0 | 0.0 | 0.0 | 0.0 | 0.0 | — |
| Cysts | 0.2 | 0.7 | 0.0 | 0.0 | 0.0 | 0.0 | 0.0 | 0.0 | — |
| Total | 31.3 | 8.5 | 30.1 | 5.8 | 32.3 | 7.9 | 33.6 | 6.2 | 0.450 |
| Number of | | | | | | | | | |
| lesions | | | | | | | | | |
| Non inflamed | 18.7 | 5.3 | 18.1 | 5.0 | 18.7 | 6.3 | 21.5 | 2.4 | 0.041 |
| lesions | | | | | | | | | |
| Inflamed | 12.6 | 5.5 | 12.0 | 3.6 | 13.6 | 4.6 | 12.1 | 5.2 | 0.714 |
| lesions | | | | | | | | | |

*p-value based on one way ANOVA

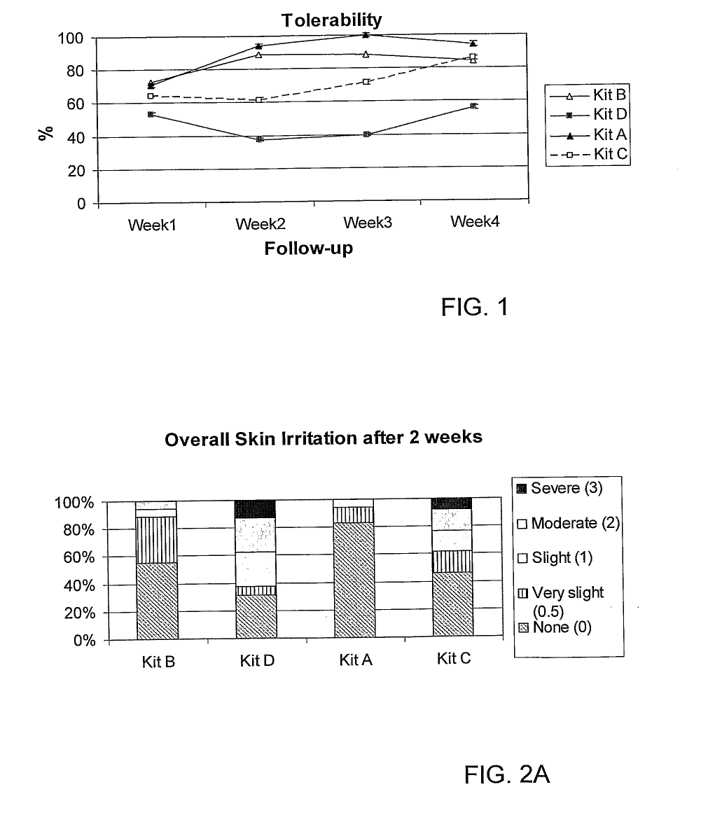

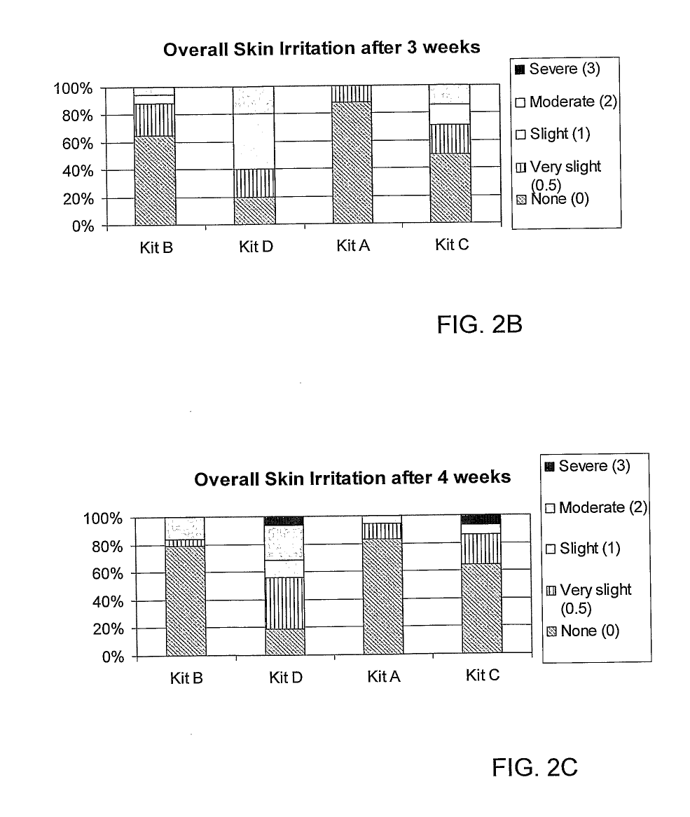

### Tolerability Assessment (FIGS. 1 and 2)

Tolerability and safety parameters were evaluated by the Investigator throughout all study visits.

The following parameters were assessed: erythema, edema, dryness, scaling/peeling, stinging/burning, itching and tightness. Each end-point was graded on a 0 to 3 scale as described.

Tolerability parameters were reported as adverse events only when un-tolerated by the patients.

The statistical analysis of the safety and tolerability parameters was done for the ITT population (all patients that received at least 1 treatment).

After 1 week of treatment—No significant difference in tolerability between the treatment kits was noted.

At week 2 Kit A was associated with significantly lower Dryness score than Kit D (p=0.002).

At week 2 Kit A was associated with significantly lower Scaling score than Kit D (p=0.006).

At week 3 Kits A and B was associated with significantly lower Dryness score than Kit D (p=0.000, p=0.004 respectively).

At week 3 Kit A was associated with significantly lower Scaling score than Kit D (p=0.003).

Although there were no significant between-groups differences, at week 3 Kit A was associated with significantly lower Dryness and Stinging scores than Kit C.

At week 4 Kits A and B were associated with significantly lower Dryness score than Kit D (p=0.000, p=0.004 respectively).

At week 4 Kit A was associated with significantly lower Scaling score than Kit D (p=0.001).

At week 2 Kit A was associated with significantly lower cumulative skin irritation scores than Kit D (p=0.002).

At week 3 Kits A and B were associated with significantly lower cumulative skin irritation scores than Kit D (p=0.001, p=0.007 respectively).

At week 4 Kits A and B were associated with significantly lower cumulative skin irritation scores than Kit D (p=0.003, p=0.008 respectively).

Although there were no significant between-groups differences, as of week 2 and throughout the entire treatment period Kits A and B were associated with lower cumulative skin irritation scores than Kit C.

At weeks 3&4 Kits A and B were associated with significantly lower skin irritation scores than Kit D (All p values >0.008).

### Efficacy Results

### Lesions Count

Efficacy evaluation was based on counting the non-inflamed, inflamed and total number of lesions at each of the 6 visits.

Non-inflamed lesions included open (blackhead) and closed (whitehead) comedones. Inflamed lesions included papules, pustules and nodulocystic lesions.

The Percent improvement of each week compared to baseline was calculated for each patient and than averaged per group.

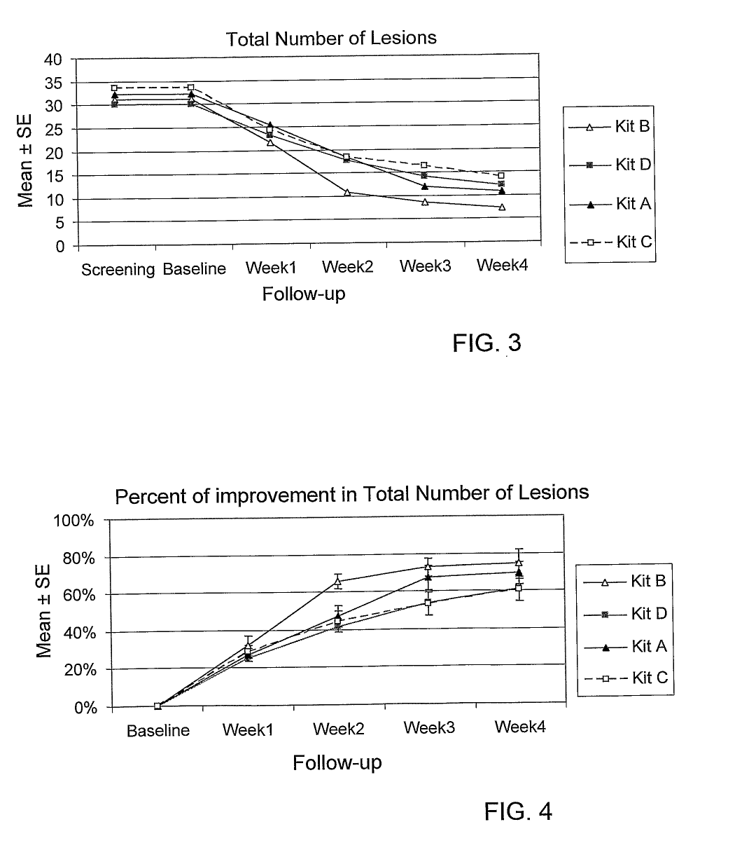

Total Number of Lesions (FIGS. 3 and 4)

At week 2 there was a significantly greater percentage change in Kit B (65.6%) as compared with Kit D (41.7%) & Kit C (44.6%) (p=0.020, p=0.033 respectively).

At week 4 and during the entire treatment period there was a significantly greater percentage change in Kit B (74.9%) as compared with Kit D (61.2%) & Kit C (60.5%) (v0.047, p=0.030 respectively).

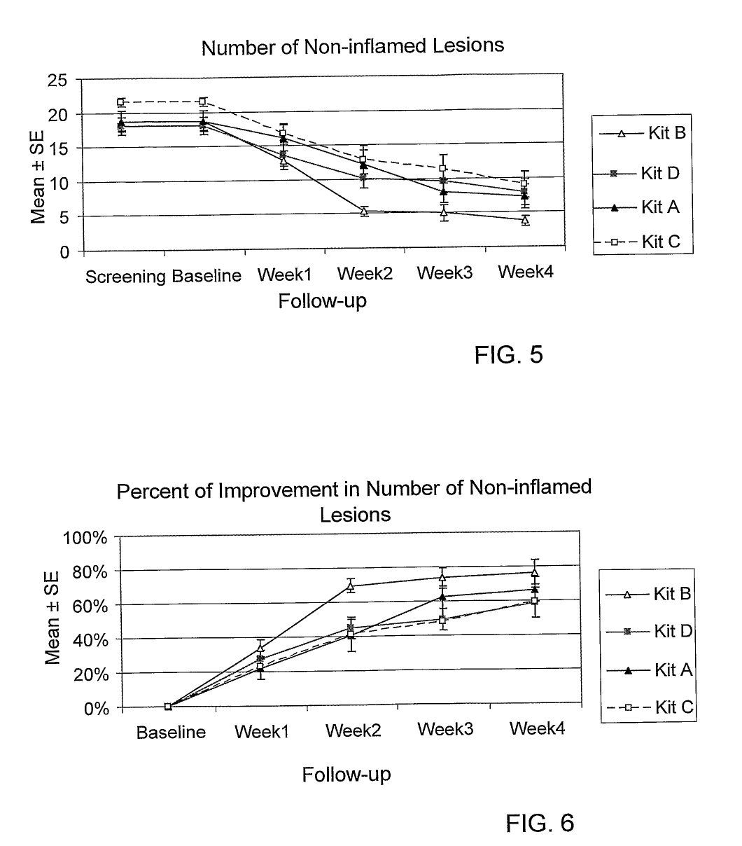

Non-Inflamed Lesions (FIGS. 5 and 6)

At week 2 there was a significantly greater percentage change in Kit B (69.6%) as compared with Kit D (45.1%) & Kit C (41.0%) (p=0.016, p=0.033 respectively) and a greater percentage change as compared with Kit A (40.4%).

At week 4 and during the entire treatment period there was a significantly greater percentage change in Kit B (76.4%) as compared with Kit A (66.5%) and Kit D (58.8%) & Kit C (59.3%) (p=0.052, p=0.010, p=0.021 respectively).

Additional analysis was performed on black and white comedones separately.

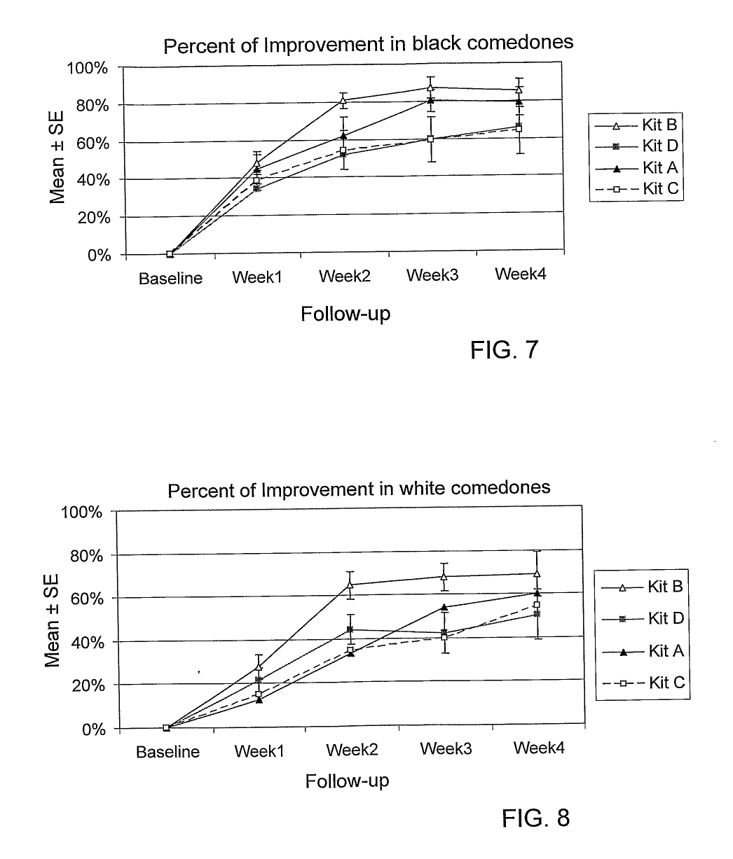

Black Comedones (FIG. 7)

Although there were no significant between-groups differences, at week 2 there was a greater decrease in black comedones by Kit B (80.9%) compared to Kit D (51.8%) C (54.4%).

At week 4 there was a greater decrease in black comedones by Kit B (85.5%) and A (79.4%) compared to Kit D (66.0%) & C (64.2%).

White Comedones (FIG. 8)

Although there were no significant between-groups differences, at week 2 there was a greater decrease in black comedones by Kit B (64.6%) compared to Kit D (44.3%) & C (34.7%).

At overall comparison there was a significantly greater decrease in black comedones by Kit B compared to Kit A and Kit D & C (p=0.040, p=0.040, p=0.008 respectively).

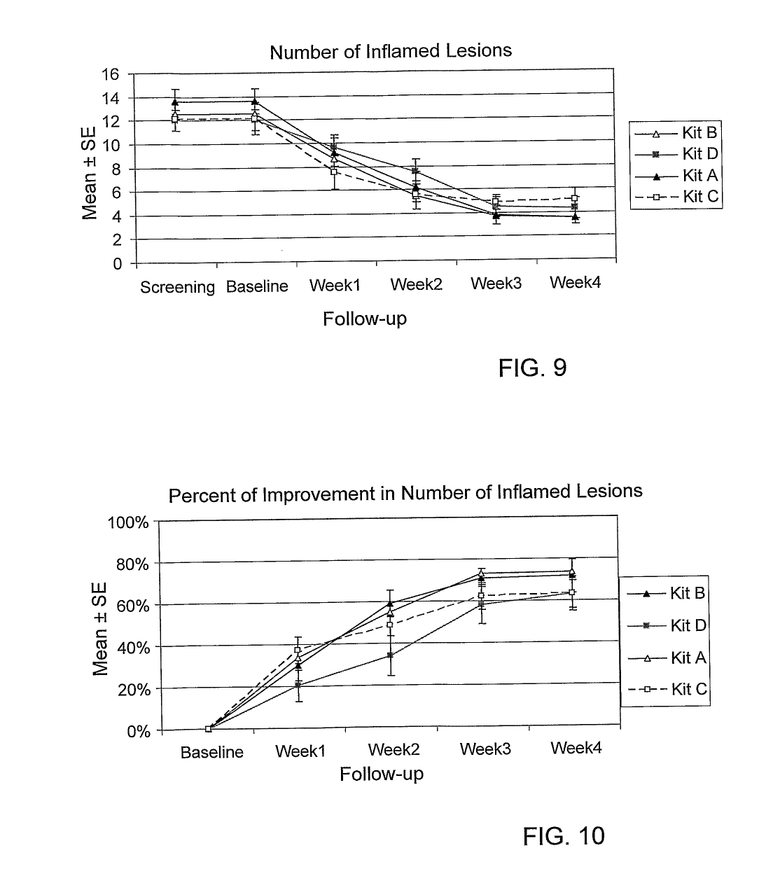

Inflamed Lesions (FIGS. 9 and 10)

Although there were no significant between-groups differences, at week 4 there was a greater percentage change in Kit B (71.6%) and A (73.8%) as compared with Kit D (63.1%) & C (63.0%)

### Investigator's Global Assessment (IGA)

Efficacy was also evaluated by the Investigator's Global Assessment

Although there were no significant between-groups differences, at week 2 there was a greater percentage (compared to week 1) in Kit B (52.9%) and A (41.2%) as compared with Kit D (13.3%) & C (15.4%)

The IGA score increased after the third and fourth weeks of treatment.

Although there were no significant between-groups differences, at week 4 there was a greater percentage (compared to week 3) in Kit B (72.2%) and A (82.4%) as compared with Kit D (60.0%) and C (35.7%).

At week 4 only Kit B showed excellent improvement (11%).

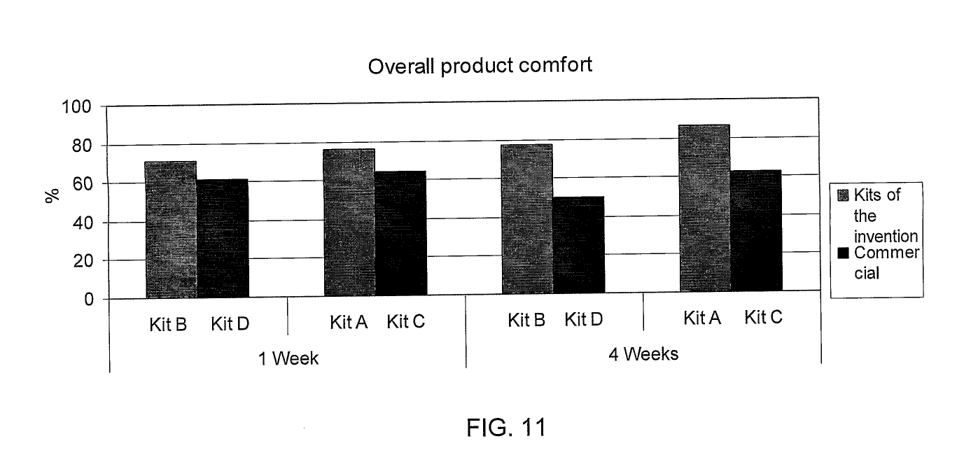

Consumer Questionnaires (FIG. 11)

Each patient completed treatment satisfactory a questionnaire during visits 3 and 6 (after 1 week and 4 weeks of treatment). Indicating parameters related to the level of his satisfaction of the treatment.

After 1 week of treatment the highest satisfaction results were achieved for Kit B and A:

Although there were no significant between-groups differences, at week 1 patients ratings from “Marked improvement of skin redness” was higher with Kit B (22%) and A (24%) than Kit D (7.1%) and C (7.1%).

Although there were no significant between-groups differences, at week 1 patients ratings from “Marked satisfaction in Kit's effectiveness” was the highest with Kit A (70.6%).

Although there were no significant between-groups differences, at week 1 patients ratings from “Improvement noted within 3 days from first use” was the highest with Kit A (87.5%).

Although there were no significant between-groups differences, at week 1 patients ratings from “Reduces blackheads” was the highest with Kit A (92.9%).

Although there were no significant between-groups differences, at week 1 patients ratings from “Does not cause stinging sensation” was the highest with Kit A (76.5%).

After 4 weeks of treatment Kits B and A continued to demonstrate higher satisfaction results:

Although there were no significant between-groups differences, at week 4 patients ratings from “Marked improvement in Number of lesions” was the highest with Kit B (78.9%).

Although there were no significant between-groups differences, at week 4 patients ratings from “Marked improvement in Overall skin look” was the highest with Kit B (84.2%).

At week 4 patients ratings from “Gentle and not irritating upon application” was the highest with Kit B (89.5%) and Kit A (83.3%) compared with Kit D (50.0%) and Kit C (64.3%) (p=0.038).

At week 4 patients ratings from “Does not cause stinging sensation” was the highest with Kit B (78.9%) and Kit A (83.3%) compared with Kit D (43.8%) and Kit C (57.1%) (p=0.001).

At week 4 patients ratings from “Does dry out the skin” was the highest with Kit B (68.4%) and Kit C (94.4%) compared with Kit D (31.3%) and Kit A (50.0%) (p=0.047).

Conclusions

The efficacy of the treatment was measured by three different parameters; count of lesions (total, non-inflamed and inflamed), Investigator's Global Assessment (IGA) and subjects' reported satisfaction from the treatment (by Consumer Questionnaires).

In spite of the relatively small sample size of the 4 treatment groups, a clinically substantial and statistically significant difference between the Kits A and B and the Kits C and D was noted.

The Kit B exhibited a statistically significant reduction in the total number of lesions as compared the two Kit D and C treatment groups. The marked differences were seen after two weeks of treatment and lasted until the end of the study.

Differences between the Kits A and B and Kits C and D were also noted by the IGA score (investigator's overall evaluation of improvement). Both kits A and B exhibited higher percentage of improvement. These differences were not statistically significant.

The comparison between the results of study subjects questionnaires by the 4 treatment groups indicated that the Kits A and B were perceived as significantly more effective in reducing the number of acne lesions and achieving improved overall skin look (Kit B).

Other parameters that showed higher percentage of satisfaction included the following claims:

Gentle and not irritating (89.5% of Kit B treatment versus 50% of Kit D),

Does not cause stinging (83.3% of Kit B streatment versus 64.3% of Kit C)

Did not dry out the skin (94.4% of Kit A treatment versus 50% of Kit C).

A comparison between the different tolerability parameters among the 4 treatment groups showed that both Kits A and B were better tolerated than kits C and D.

After two weeks of treatment the rate and severity of dry skin and scaling were perceived by the subjects as significantly lower for the Kit A versus the Kit C (0.002 for dry skin and 0.006 for scaling) and after 4 weeks of treatment the rate and severity of dry skin and scaling between the Kit B and the Kit D was 0.007 for dry skin and 0.023 for scaling respectively.

The efficacy and tolerability results of this study indicate that subjects with mild and moderate acne can benefit from using Kits A and B more effectively and safely than using prior art kits C and D, by having less adverse reactions and better improvement of acne lesions.

1.-49. (canceled)

50. A multi component topical system, comprising:
at least one component comprising at least one first active agent and coated on the at least one first active agent; and at least one skin treating component free of the active agent, wherein each of the components is encompassed in a different vessel or container.

51. The system according to claim 50, wherein the at least one first active agent coated by metal oxide provides gradual release of the at least one first active agent.

52. The system according to claim 50, further comprising at least one further component comprising at least one further active agent and a metal oxide coated on the at least one further active agent.

53. The system according to claim 50, wherein the metal oxide coating of the active agent is obtained by a process, comprising:
(a) contacting a water-insoluble active agent with an ionic additive in an aqueous medium to obtain a dispersion of active agent particulates having charged surface;
(b) precipitating a metal oxide salt onto the surface of the active agent particulates to form a metal oxide coat on the particulates;
(c) optionally repeating step (b); and
(d) aging the metal oxide coating on the particulates.

54. The system according to claim 50, wherein the metal oxide coating of the active agent is obtained by a process, comprising:
(a) contacting at least one water-insoluble active agent with a cationic additive in an aqueous medium to obtain a dispersion of active agent particulates having positive charges on at least a portion of their surface;
(b) precipitating a metal oxide salt onto at least a portion of the surface of the active agent particulates to form a metal oxide coating on the particulates;
(b1) contacting the metal oxide coated active agent particulates with at least one additive selected from (i) a further cationic additive, and (ii) a non-ionic additive, thereby obtaining a metal oxide coated active agent particulates having the at least one additive on at least a portion of particulates' surface;
(b2) precipitating a metal oxide salt onto at least a portion of the surface of the metal oxide coated active agent particulates having at least one additive on at least a portion of the particulates' surface to form a further metal oxide coating on the particulates;
(c) optionally repeating steps (b1) and (b2); and
(d) aging the metal oxide coating on the particulates.

55. The system according to claim 50, wherein the metal oxide coating of the active agent is obtained by a process, comprising:
(a) contacting at least one water-insoluble active agent, with an anionic additive, a cationic additive in an aqueous medium to obtain a dispersion active agent particulates having positive charges on at least a portion of their surface;
(b) precipitating a metal oxide salt onto at least a portion of the surface of the active agent particulates to form a metal oxide coating on the particulates;
(b1) contacting the coated active agent particulates with at least one additive selected from (i) a further cationic additive, and (ii) a non-ionic additive, thereby obtaining coated active agent particulates having the at least one additive on at least a portion of their surface;
(b2) precipitating a metal oxide salt onto at least a portion of the surface of the metal oxide coated active agent particulates having at least one additive on at least a portion of the particulates' surface to form a further metal oxide coating on the particulates;
(c) optionally repeating steps (b1) and (b2); and
(d) aging the metal oxide coating on the particulates.

56. The system according to claim 50, wherein the at least one active agent is selected from the group consisting of an anti fungal agent, an anti bacterial agent, an anti viral agent, an anti inflammatory agent, an anti pruritic agent, an anti psoriatic agent, an anti seborrhea agent, an anti acne agent, an anti rosacea agent, an anti cancer agent, an anti photoaging agent, a skin whitening/bleaching agent, an anti skin redness agent, and any combinations of the above.

57. The system according to claim 50, wherein the at least one active agent is benzoyl peroxide.

58. The system according to claim 50, wherein the metal oxide is selected from the group consisting of Silica, Titania, Alumina, Zirconia, ZnO, and mixtures thereof.

59. The system according to claim 50, wherein the metal oxide coating of the active agent has a width of 0.1-10 micron.

60. A method for treating, improving appearance of or preventing a disease or condition of the skin or of a mucosal membrane, comprising topically administering onto the skin or mucosal membrane a multi component topical system comprising at least one component comprising at least one first active agent and a metal oxide coated on the at least one first active agent; and at least one skin treating component free of the active agent, wherein each of the components is encompassed in a different vessel or container.

61. The method according to claim 60, wherein the at least one first active agent coated by metal oxide provides gradual release of the at least one first active agent.

62. The method according to claim 60, wherein the skin or mucosal membrane disease or condition is a disease or disorder selected from the group consisting of acne, infection, inflammation, pruritis, psoriasis, seborrhea, contact dermatitis, skin cancer, skin photoaging, pigmentation, redness, rosacea, and any combination thereof.

63. The method according to claim 60, wherein the skin or mucosal membrane disease or condition is skin lesions.

64. The method according to claim 60, wherein the skin or mucosal membrane disease or condition is skin comedones.

65. The method according to claim 60, wherein the skin or mucosal membrane disease or condition is acne.

66. The method according to claim 60, wherein system components are administered in a pre-determined administration schedule.

67. A kit, comprising:
at least one component comprising at least one first active agent and a metal oxide coated on the at least one first active agent; and at least one skin treating component free of the active agent, wherein each of the components is encompassed in a different vessel or container; and instructions for use.

68. The kit according to claim 67, wherein the at least one first active agent coated by metal oxide provides gradual release of the at least one first active agent.

69. The kit according to claim 67, comprising at least one further component comprising at least one further active agent and a metal oxide coated on the at least one further active agent.
